(19) United States
(12) Patent Application Publication
(10) Pub. No.: US 2004/0242460 A1
(43) Pub. Date: Dec. 2, 2004

(54) Stabilized acylated insulin formulations

(76) Inventors: Mark Laurence Brader, Indianapolis, IN (US); Michael John Beckage, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/781,393

(22) Filed: Feb. 18, 2004

Related U.S. Application Data
(63) Continuation of application No. 08/484,542, filed on Jun. 7, 1995, now abandoned.

(51) Int. Cl. A01N 37/18; A61K 38/00

(52) U.S. Cl. 514/2; 530/300

(57) ABSTRACT

A storage stable formulation comprising an aqueous solution suitable for parenteral delivery, particularly as an injectable formulation to a patient, preferably having a pH of 7.1 to 7.6, containing a fatty acid-acylated insulin or a fatty acid-acylated insulin analog and stabilized using zinc and preferably a phenolic compound.

### BACKGROUND OF THE INVENTION

1. Field of the Invention

The present invention is broadly directed to the preparation of stable formulations containing certain recently developed acylated insulins, and especially formulations suitable for parenteral delivery, particularly as an injectable formulation, to a patient. More particularly, the present invention relates to the preparation of storage stable aqueous formulations of certain acylated insulins and insulin analogs using zinc as a necessary ingredient and preferably including a phenolic compound.

2. Description of Related Art

It has long been a goal of insulin therapy to mimic the pattern of endogenous insulin secretion in normal individuals. The daily physiological demand for insulin fluctuates and can be separated into two phases: (a) the absorptive phase requiring a pulse of insulin to dispose of the meal-related blood glucose surge, and (b) the post-absorptive phase requiring a sustained amount of insulin to regulate hepatic glucose output for maintaining optimal fasting blood glucose. Accordingly, effective therapy generally involves the combined use of two exogenous insulins: a fast-acting meal time insulin provided by bolus injections and a long-acting basal insulin administered by injection once or twice daily.

Recently, a class of acylated insulins has shown promise for use as a long-acting basal insulin therapy. These acylated insulins are prepared by acylating, selectively with an activated fatty acid derivative, the free amino group(s) of a monomeric insulin, including normal insulin and certain insulin analogs. Useful fatty acid derivatives include reactive fatty acid-type compounds having at least a six (6) carbon atom chain length and particularly those fatty acid derivatives having 8 to 21 carbon atoms in their chain. Mono-acylated normal human insulin, acylated with a palmitic acid derivative, is a particularly promising candidate. Insulins falling within this category are described in Japanese patent application 1-254,699.

As well-understood by those skilled in this art, normal insulin has long been prepared in storage stable aqueous formulations by combining it with zinc and a phenolic preservative. Absent such stabilizers, insulin solutions exhibit a tendency to produce aggregates or precipitates and to experience a reduction in potency. Current long-acting insulin preparations, in contrast, are provided as suspensions or emulsions of the active insulin component in an aqueous vehicle. A long-acting, soluble form of insulin, which is stable at a physiological pH, would have advantages.

Like normal insulin, certain acylated insulins, and in particular those insulins acylated with the above-mentioned long chain fatty acid derivatives, have also been found to be unstable upon prolonged storage in aqueous solution. Storage instability is a particular problem with acylated Biosynthetic Human Insulin (BHI), wherein the ε amino group of Lys^{B29} is acylated with a palmitic acid derivative, i.e., N-palmitoyl Lys^{B29} human insulin. This acylated insulin also shows a strong tendency to form higher aggregates and thus has a much more limited solubility compared to normal BHI. In order to provide formulations of such acylated insulins convenient for mass distribution, especially in a soluble form suitable for parenteral delivery to a patient, a storage stable preparation of these acylated insulins is needed.

The present invention provides a simple formulation containing such fatty acid-acylated insulins and insulin analogs that exhibit sufficient chemical and physical stability, particularly with respect to solubility and potency, needed to survive extended storage associated with mass distribution of pharmaceuticals.

### DESCRIPTION OF THE INVENTION

All amino acid abbreviations used in this disclosure are those accepted by the United States Patent and Trademark Office as set forth in 37 C.F.R. § 1.822(B)(2).

The terms “insulin” and “normal insulin” as used herein mean human insulin, pork insulin, or beef insulin. Insulin possesses three free amino groups: B¹-Phenylalanine, A¹-Glycine, and B²⁹-Lysine. The free amino groups at positions A¹ and B^{l} are α-amino groups. The free amino group at position B²⁹ is an ε-amino group.

The term “proinsulin” as used herein is a properly cross-line protein of the formula:

B-C-A

wherein:

A is the A chain of insulin or a functional derivative thereof;

B is the B chain of insulin or a functional derivative thereof having an ε-amino group; and

C is the connecting peptide of proinsulin. Preferably, proinsulin is the A chain of human insulin, the B chain of human insulin, and C is the natural connecting peptide. When proinsulin is the natural sequence, proinsulin possesses three free amino groups: Phenylalanine(1) (α-amino group), Lysine(29) (ε-amino group) and Lysine(64) (ε-amino group).

The term “insulin analog” as used herein is a properly cross-lined protein exhibiting insulin activity of the formula:

A-B

wherein:

A is the A chain of insulin or a functional derivative of the insulin A chain; and

B is the B chain of insulin or a functional derivative of the insulin B chain having an ε-amino group and at least one of A or B contains an amino acid modification from the natural sequence.

Preferred insulin analogs include insulin wherein:

the amino acid residue at position B²⁸ is Asp, Lys, Leu, Val, or Ala;

the amino acid residue at position B²⁹ is Lys or Pro;

the amino acid residue at position B¹⁰ is His or Asp;

the amino acid residue at position B¹ is Phe, Asp, or deleted alone or in combination with a deletion of the residue at position B²;

the amino acid residue at position B³⁰ is Thr, Ala, or deleted; and

the amino acid residue at position B⁹ is Ser or Asp; provided that either position B²⁸ or B²⁹ is Lys.

In standard biochemical terms known to the ordinarily skilled artisan the preferred insulin analogs are Lys^{B28}Pro^{B29}-human insulin (B²⁸ is Lys; B²⁹ is Pro); Asp^{B28}-human insulin (B²⁸ is Asp); Asp^{B1}-human insulin, Arg^{B31,B32}-human insulin, Asp^{B10}-human insulin, Arg^{A0}-human insulin, Asp^{B1},Glu^{B13}-human insulin, Ala^{B26}-human insulin, and Gly^{A21}-human insulin.

The term “acylating” means the introduction of one or more acyl groups covalently bonded to the free amino groups of the protein.

The term “fatty acid” means a saturated or unsaturated C₆-C₂₁ fatty acid. The term “activated fatty acid ester” means a fatty acid which has been activated using general techniques described in Methods of Enzymology, 25:494-499 (1972) and Lapidot et al., in J. of Lipid Res, 8:142-145 (1967), incorporated herein by reference. The preferred fatty acids are saturated and include myristic acid (C₁₄), pentadecylic acid (C₁₅), palmitic acid (C₁₆), heptadecylic acid (C₁₇) and stearic acid (C₁₈). Most preferably, the fatty acid is palmitic acid. Activated fatty acid ester includes derivatives of agents such as hydroxybenzotriazide (HOBT), N-hydroxysuccinimide and derivatives thereof. The preferred activated ester is N-succinimidyl palmitate.

The term “cross-link” means the formation of disulfide bonds between cysteine residues. A properly cross-linked proinsulin, insulin or insulin analog contains three disulfide bridges. The first disulfide bridge is formed between the cysteine residues at positions 6 and 11 of the A-chain. The second disulfide bridge links the cysteine residues at position 7 of the A-chain to the cysteine at position 7 of the B-chain. The third disulfide bridge links the cysteine at position 20 of the A-chain to the cysteine at position 19 of the B-chain.

The term “aqueous” includes cosolvent systems as well as use of water only as a solvent. Aqueous compositions containing water as the major, if not the sole, solvent are preferred.

The present invention relates to the preparation of storage stable formulations of certain fatty acid-acylated insulins and insulin analogs. In one particularly preferred aspect, the present invention relates to a composition comprising an aqueous solution of a fatty acid-acylated insulin, a source zinc cations, and preferably, though optionally a phenolic compound.

Insulin and insulin analogs used to prepare the fatty acid-acylated insulins that are the principal focus of the present invention can be prepared by any of a variety of recognized peptide synthesis techniques including classical (solution) methods, solid phase methods, semi-synthetic methods, and more recent recombinant DNA methods. For example, Chance et al., U.S. patent application Ser. No. 07/388,201, EPO publication number 383 472, Brange et al., EPO 214 826, and Belagaje et al., U.S. Pat. No. 5,304,473 disclose the preparation of various proinsulin and insulin analogs and are herein incorporated by reference. The A and B chains of the insulin analogs of the present invention may also be prepared via a proinsulin-like precursor molecule using recombinant DNA techniques. See Frank et al., Peptides: Synthesis-Structure-Function, Proc. Seventh Am. Pept. Symp., Eds. D. Rich and E. Gross (1981) which is incorporated herein by reference. The source of the insulin is not critical, though insulins having the structure of that produced by humans and the insulin analog Lys²⁸Pro^{B29} human insulin are preferred.

Generally, the insulin and insulin analogs are acylated by reacting them with an activated fatty acid derivative, such as an activated fatty acid ester. The acylation of normal insulin with a fatty acid is disclosed in Japanese patent application 1-254,699. See also Hashimoto et al., Pharmaceutical Research, 6: 171-176 (1989). These disclosures are incorporated herein by reference.

Preferably, the acylation is conducted under basic conditions, i.e., at a pH greater than 9.0 and preferably about 10.5, in a polar solvent. While the reaction can be conducted in a wholly organic polar solvent using a base having an aqueous pKa of greater than or equal to 10.75, a mixed organic and aqueous solvent is generally preferred for the reaction medium. Preferred bases are tetramethylguanidine, diisopropylethylamine or tetrabutylammonium hydroxide. One particularly suitable solvent has been acetonitrile and water, containing about 50% acetonitrile. Other polar solvents include dimethyl sulfoxide, dimethylformamide and the like. Cosolvents also include acetone and water, isopropyl alcohol and water, and ethanol and water. Time and temperature conditions suitable for conducting the reactions are not narrowly critical. A temperature of 0 to 40° C. and a reaction time of 15 minutes to 24 hours should generally be suitable. A particularly preferred way of preparing such fatty acid-acylated insulins is described in copending U.S. application Ser. No. 08/341,231 filed Nov. 17, 1994, the disclosure of which is incorporated herein by reference.

Once the reaction is complete, the reaction mixture typically is diluted with water and an acid is added to neutralize the alkalinity. The acid is supplied as an aqueous solution to the acylated protein and serves to lower the solution pH to below the isoelectric point of the protein. Normally, at this point the protein is in a properly buffered aqueous solution for further processing. Such processing particularly includes purification by standard methods such as reverse phase or hydrophobic chromatography and concentration by ultrafiltration. For acylated insulin and acylated insulin analogs, particularly N-acylated Lys^{B29} human insulin and B28-N^{ε}-acylated-Lys^{B28}Pro^{B29}-human insulin and especially the palmitic acid-acylated species, the pH normally should be adjusted to below about 3.0, and preferably to between about 1.5 and 2.5, using the acid as-needed. Suitable acids include HCl, acetic acid, glycine and citric acid. Use of citric acid at a concentration of 50 mM has been found suitable. If needed the pH also can be readjusted with a base, such as sodium hydroxide, to keep it within the desired limits.

At this point, the aqueous solution of the purified acylated protein, particularly a fatty acid-acylated insulin or a fatty acid-acylated insulin analog, can be processed to recover the soluble protein as a powder. In the broad practice of the present invention, any procedure for recovering the acylated protein as a powder, including lyophilization (freeze drying) crystallization or precipitation techniques, can be used. The present invention is not limited to the way of isolating and recovering the acylated insulin or insulin analog in powder form.

The acylated insulin or acylated insulin analog powder can then be used to prepare the storage stable formulations of the present invention useful for insulin therapy, i.e. for administering to a patient in need thereof (i.e. a patient suffering from hyperglycemia). Such formulations contain an effective amount of the fatty acid-acylated insulin in combination with the required stabilizing ingredient(s) and normally with one or more pharmaceutically acceptable excipients or carriers.

In one aspect of the present invention, an aqueous solution of the purified fatty acid-acylated insulin, and especially an aqueous solution of N-palmitoyl Lys^{B29} human insulin or an aqueous solution of a purified fatty acid-acylated insulin analog and especially B28-N^{ε}-palmitoyl-Lys^{B28}Pro^{B29}-human insulin, is fortified with zinc prior to lyophilization. Typically, a water soluble zinc salt, such as zinc chloride or zinc acetate, is added to and mixed with an aqueous acylated protein solution in an amount of 0.2 mole zinc per mole of insulin and up to 0.7 mole zinc per mole of insulin. Preferably the zinc is added in an amount of 0.3 to 0.55 mole per mole of insulin. Applicants have found that lyophilizing an aqueous solution of the fatty-acid acylated insulin in the presence of zinc enhances protein stability, compared to zinc-free lyophilized powder.

The present invention especially pertains to storage stable aqueous solutions of the fatty-acid acylated insulin and fatty acid-acylated insulin analog. In such solutions the acylated insulin or insulin analogs, and particularly N-palmitoyl Lys^{B29} human insulin may be present in varying concentrations ranging from about 1.4 mg/ml to about 11 mg/ml. For N-palmitoyl Lys^{B29} human insulin, a concentration in the range of about 2.9 to 3.8 mg/ml will be suitable in most cases. These compositions are typically, though not necessarily, parenteral in nature.

In accordance with the present invention, zinc is present in the formulation in an amount of from about 0.2 mole to about 0.7 mole per mole of the fatty acid-acylated insulin and fatty acid-acylated insulin analog, preferably about 0.30 to 0.55 mole of zinc per mole of acylated insulin and most preferably about 0.35 mole zinc per mole of acylated insulin. For N-palmitoyl Lys^{B29} human insulin, this corresponds to an amount of zinc in the composition broadly from about 0.22% to about 0.76% by weight of the acylated insulin, preferably from about 0.38% to about 0.59% by weight based upon the acylated insulin content of the formulation and most preferably about 0.38% by weight. Conveniently, the zinc can be added as one of its water-soluble salts such as zinc chloride, zinc acetate and the like.

Another preferred stabilizing component of the aqueous formulation of this invention is a phenolic compound. When used, the phenolic compound is present broadly in an amount of from about 0.5 mg. to about 5 mg. per each milliliter of the aqueous formulation (about 0.05% to about 0.5% by weight). Preferably, the phenolic compound is present in an amount to also provide the composition with a preservative effect. In this regard, the phenolic compound typically would be present in an amount of at least about 2.5 mg/ml of solution up to about 5.0 mg/ml. Most commercial preparations will contain a phenolic compound in an amount ranging from about 2.75 mg to about 3.2 mg per each milliliter of the formulation. Suitable phenolic compounds include, for example phenol, m-cresol, o-cresol, p-cresol and methylparaben. Preferred phenolic compounds are phenol and m-cresol. When using phenol it is preferred to use about 2.75 mg/ml, with m-cresol about 3.15 mg/ml. Mixtures of phenolic compounds also are contemplated, and a mixture of 2.15 mg/ml of m-cresol with 0.87 mg/ml of phenol is suitable.

The injectable formulations of the present invention can be prepared using conventional dissolution and mixing procedures. To prepare a suitable formulation, for example, a measured amount of the fatty acid-acylated insulin or fatty acid-acylated insulin analog powder is combined with the other required ingredients, including water in an amount sufficient to dissolve the components and provide the insulin species at the desired strength and a soluble zinc salt. Preferably, a phenolic-compound also is included. Applicants have found that the acylated insulin, particularly N-palmitoyl Lys^{B29} human insulin, forms a complex with the zinc and phenolic additives, which apparently contributes to the improved chemical stability observed with these formulations.

At this point, it also is important to adjust the pH of the solution to maximize product storage stability. A variety of acids, such as hydrochloric acid, acetic acid, citric acid and the like, and a variety of bases, such as sodium hydroxide, ammonium hydroxide and the like, can be used for the pH adjustment. The pH of the solution influences both the chemical stability of the insulin solution and the solubility of the insulin in the aqueous formulation. The pH should be adjusted to within the range of 6.8 to 7.8. Applicants determined that the chemical stability of aqueous solutions of acylated insulins is better at the lower end of the recited pH range, while the solubility of the acylated insulins is better at the higher end of the range. For commercial preparations of N-palmitoyl Lys^{B29} human insulin the pH of the solution preferably is in the range of 7.1 to 7.6, more preferably about 7.2 to 7.3. Thereafter, the solution can be sterilized, such as by micro filtration, and then is aseptically filled and sealed, for example in a vial.

In additional to the insulin, zinc, and the phenolic compound, pharmaceutical compositions adapted for parenteral administration in accordance with the present invention may employ, additional excipients and carriers such as water-miscible organic solvents such as glycerol, sesame oil, groundnut oil, and aqueous propylene glycol and the like. When present, such agents are usually used in an amount ranging from about 0.5% to about 2.0% by weight based upon the final formulation. Examples of such pharmaceutical compositions include sterile, isotonic, aqueous saline solutions of the insulin buffered with a pharmaceutically acceptable buffer and pyrogen free. For further information on the variety of techniques using conventional excipients or carriers for parenteral products, please see Remington's Pharmaceutical Sciences, 17th Edition, Mack Publishing Company, Easton, Pa., USA (1985) which is incorporated herein by reference.

In the broad practice of the present invention, it also is contemplated that a formulation may contain a mixture of an acylated insulin or acylated insulin analog with a normal insulin and/or an insulin analog. For example, N-palmitoyl Lys^{B29} human insulin may be mixed with Biosynthetic Human Insulin (BHI) and/or with Lys^{B28}Pro^{B29} human insulin. Such insulin mixtures would be designed to provide a desired mode of action. Such mixtures likely would contain an acylated insulin or insulin analog to normal insulin and/or insulin analog mole ratio of 30:1 to 1:3.

The following example is presented to illustrate and explain the invention. While the invention is illustrated by reference to the preparation of a stable formulation of N-palmitoyl Lys^{B29} human insulin, the scope of the invention should not be considered as being limited to this example. Unless otherwise indicated, all references to parts and percentages are based on weight and all temperatures are expressed in degrees Celsius.

### EXAMPLE

N-palmitoyl Lys^{B29} humin insulin (36.4 mg) can be dissolved in 8 mls of 0.01 N HCl. Thereafter, 0.138 mg of zinc is added as zinc chloride from a 10 mg/ml zinc chloride stock solution, prepared by dissolving zinc oxide in hydrochloric acid. The pH of the solution of acylated insulin is adjusted to 7.5 with 1N sodium hydroxide. Glycerol (160 mg) is added as an isotonic agent. m-Cresol (25 mg) then is added and the solution is mixed thoroughly. The pH is adjusted to 7.2 with 1N hydrochloric acid and 1N sodium hydroxide and the solution volume is adjusted to 10 mls with water. The solution then can be filtered and aseptically filled into a vial.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Variations and changes may be made by those skilled in the art without departing from the spirit of the invention.

1-26. (Canceled).

27. A composition comprising
(a) a fatty acid-acylated insulin or a fatty acid-acylated insulin analog in which the amino acid residue at position B30 is Thr, Ala or deleted, and
(b) zinc.

28. A composition comprising an aqueous solution of
(a) a fatty acid-acylated insulin or a fatty acid-acylated insulin analog in which the amino acid residue at position B30 is Thr, Ala or deleted, and
(b) zinc.

29. The composition of claim 28, wherein the solution comprises about 0.2 mole to about 0.7 mole of zinc per mole of fatty acid-acylated insulin or fatty acid-acylated insulin analog.

30. The composition of claim 29, wherein the pH is 6.8 to 7.8.

31. The composition of claim 30, further comprising a phenolic compound at a concentration of from 0.5 mg to 5 mg per milliliter of the aqueous solution.

32. The composition of claim 31, wherein the fatty acid-acylated insulin is N-acylated Lys^{B29} human insulin, and wherein the fatty acid-acylated insulin analog is an N-acylated Lys^{B29} insulin analog in which the amino acid residue at position B30 is Thr, Ala or deleted.

33. The composition of claim 28, wherein the composition is a pharmaceutical composition that further comprises a phenolic compound, glycerol, and a pharmaceutically acceptable buffer.

34. The composition of claim 27, wherein the composition comprises about 0.2 mole to about 0.7 mole of zinc per mole of fatty acid-acylated insulin or fatty acid-acylated insulin analog.

35. The composition of claim 27, wherein the fatty acid-acylated insulin is N-acylated Lys^{B29} human insulin, and wherein the fatty acid-acylated insulin analog is an N-acylated Lys^{B29} insulin analog in which the amino acid residue at position B30 is Thr, Ala or deleted.

36. The composition of claim 32, wherein the fatty acid-acylated insulin is N-myristoyl Lys^{B29} human insulin, and wherein the solution comprises from about 0.3 mole to about 0.55 mole of zinc per mole of fatty acid-acylated insulin.

37. The composition of claim 36, wherein the concentration of phenolic compound is from about 2.5 mg to about 5.0 mg per milliliter of the aqueous solution.

38. The composition of claim 37, wherein the phenolic compound is selected from the group consisting of phenol, m-cresol, p-cresol, o-cresol, methylparaben, and mixtures thereof.

39. The composition of claim 38, wherein the phenolic compound is selected from the group consisting of phenol and m-cresol.

40. The composition of claim 27, wherein the fatty acid in the fatty acid-acylated analog is myristic acid.

41. The composition of claim 28, wherein the fatty acid in the fatty acid-acylated analog is myristic acid.

42. The composition of claim 34, wherein the pH is 6.8 to 7.8.
